(19) United States
(12) Patent Application Publication
(10) Pub. No.: US 2024/0302280 A1
(43) Pub. Date: Sep. 12, 2024

(54) PREPARATION METHOD AND APPLICATION OF VISCOSITY FLUORESCENT PROBE

(71) Applicant: Zhengzhou University of Light Industry, Zhengzhou (CN)

(72) Inventors: Hua Zhang, Zhengzhou (CN); Keke Zhang, Zhengzhou (CN); Yizhe Yan, Zhengzhou (CN); Jianyong Wang, Zhengzhou (CN); Shaoqing Li, Zhengzhou (CN); Xiaopei Zhu, Zhengzhou (CN)

(21) Appl. No.: 18/589,519

(22) Filed: Feb. 28, 2024

(30) Foreign Application Priority Data
Mar. 6, 2023 (CN) 2023102073203

(51) Int. Cl. G01N 21/64; C07D 251/24; C09K 11/06

(52) U.S. Cl. CPC G01N 21/6486; C07D 251/24; C09K 11/06; G01N 21/6456; C09K 2211/1059; G01N 2021/6439

(57) ABSTRACT

The present invention belongs to the field of organic small molecule fluorescent probes, and in particular to a preparation method and application of a viscosity fluorescent probe. A viscosity fluorescent probe TzAr-Nap-OH based on 1,3,5 triazine dye, which shows very strong sensitivity to viscosity, has little effect on fluorescence intensity over a wide pH range and good cytocompatibility, and responds well to viscosity. In cell imaging experiments, the fluorescent probe has low cytotoxicity and may accurately locate cells, may be applied to detect cell viscosity of complex organisms, and the synthesis steps of the probe are simple with a wide range of raw material sources, which provides an available tool for studying the important role that cell viscosity plays in complex physiological processes of organisms. Therefore, the cell viscosity fluorescent probe with high sensitivity and low interference and toxicity has broad application prospects in the field of biomolecular detection.

### TECHNICAL FIELD

The present invention belongs to the field of organic small molecule fluorescent probes, and in particular to a preparation method and application of a viscosity fluorescent probe.

### BACKGROUND

Cell viscosity plays an extremely important role in the transport of various molecules in vivo, and it also affects the diffusion of biological processes, such as cell apoptosis and the transport of subcellular organelles in blood. It turns out that abnormal cell viscosity may cause a variety of diseases, such as arteriosclerosis, Alzheimer's disease, diabetes, etc. Given that changes in cell viscosity are associated with many diseases, the development of cell viscosity-responsive sensors and the development of biological research and medical diagnostic strategies have attracted great attention.

In recent years, instruments for measuring liquid viscosity have emerged in an endless stream, such as falling ball viscometers, rotational viscometers, piston viscometers, etc. However, the use of these viscometers is complicated and has shortcomings such as long use time, and thus cannot be used to detect viscosity changes at the cellular level. With the development of fluorescence imaging technology, it is possible to implement visualization by converting biological information into detectable signals. Compared with traditional viscometers, the fluorescence imaging technology has advantages such as short time, easy operation, high sensitivity, strong specificity, cell imaging, and direct imaging with confocal microscope, and it has become one of the most powerful tools for biological testing. Interestingly, there are many viscosity fluorescent probes reported so far, but there are almost no viscosity fluorescent probes based on 1,3,5 triazine dyes, and pH-resistant viscosity probes are also rare.

### SUMMARY

An object of the present invention is to provide a viscosity fluorescent probe TzAr-Nap-OH based on 1,3,5 triazine dye. The probe shows strong sensitivity to viscosity, has little effect on fluorescence intensity over a wide pH range, has good cytocompatibility, and responds well to viscosity.

The present invention adopts the following technical solution to synthesize an organic small molecule fluorescent probe that may detect viscosity.

The molecular structural formula of the viscosity fluorescent probe synthesized in the present invention is as follows:

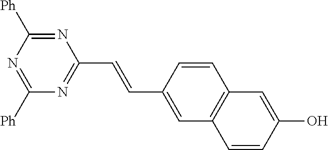 [SMILES: Oc1ccc2cc(/C=C/c3nc(-c4ccccc4)nc(-c4ccccc4)n3)ccc2c1]

The synthesis route of the probe TzAr-Nap-OH is as follows:

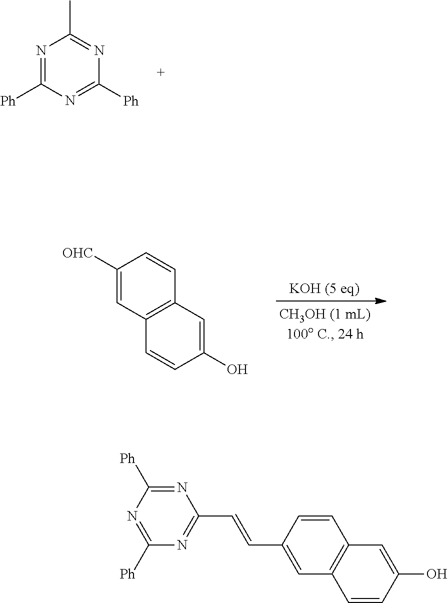 [SMILES: Cc1nc(-c2ccccc2)nc(-c2ccccc2)n1 ; O=Cc1ccc2cc(O)ccc2c1 ; Oc1ccc2cc(/C=C/c3nc(-c4ccccc4)nc(-c4ccccc4)n3)ccc2c1]

2,4-diphenyl-6-methyl-1,3,5-triazine (0.2 mmol), p-hydroxynaphtaldehyde (0.2 mmol), potassium hydroxide (1.0 mmol), and methanol (1.0 mL) were added in sequence into a 10 mL reaction tube and stirred at 100° C. for 24 hours. The solution was then cooled to room temperature and extracted three times with ethyl acetate. The organic phase was collected, dried over anhydrous sodium sulfate, and filtered with suction. The resulting filtrate was concentrated under reduced pressure and purified by column chromatography (dichloromethane:ethyl acetate=80:1), to obtain a yellow-green product TzAr-Nap-OH (15.15 mg, 48%).

Application of the viscosity fluorescent probe of the present invention: the fluorescent probe may be applied to detect cell viscosity of complex organisms; and fluorescence spectrum testing and cell imaging were performed on the probe.

Beneficial effects: in view of the problems currently posed for viscosity fluorescent probes, the present invention provides a viscosity fluorescent probe TzAr-Nap-OH based on 1,3,5 triazine dye. The fluorescent probe shows strong sensitivity to viscosity, has little effect on fluorescence intensity over a wide pH range, has good cytocompatibility, and responds well to viscosity. In cell imaging experiments, the fluorescent probe has low cytotoxicity and may accurately locate cells, which provides a feasible tool for studying the important role of cell viscosity in complex physiological processes of organisms. The fluorescent probe may be applied to detect cell viscosity of complex organisms, and the synthesis steps of the fluorescent probe are simple, with a wide range of raw material sources. Therefore, the present invention relates to a cell viscosity fluorescent probe with high sensitivity, low interference and low toxicity, which has broad application prospects in the field of biomolecular detection.

### BRIEF DESCRIPTION OF DRAWINGS

In order to illustrate the embodiments of the present invention or the technical solutions in the prior art more clearly, a brief description will be given below with reference to the need to be used in the embodiments. It is obvious that the drawings in the following description are only some embodiments of the present invention, and it would have been obvious for a person skilled in the art to obtain other drawings according to these drawings without involving any inventive effort.

FIG. 1 shows a 1H NMR spectrum of the probe TzAr-Nap-OH;

FIG. 2 shows a 13C NMR spectrum of the probe TzAr-Nap-OH;

FIG. 3 shows UV absorption spectra of the probe TzAr-Nap-OH in different solvents;

FIG. 4 shows fluorescence emission spectra of the probe TzAr-Nap-OH in different solvents;

FIG. 5 shows fluorescence emission spectra of the probe TzAr-Nap-OH under different methanol/glycerol ratios;

FIG. 6 shows fluorescence emission spectra of the probe TzAr-Nap-OH at different pH;

FIG. 7 shows fluorescence emission spectra of the probe TzAr-Nap-OH in the presence of other interfering substances; 1, Arg; 2, H2O2; 3, Ser; 4, HClO; 5, NaCl; 6, Ba2+; 7, Zn2+; 8, KI; 9, SO32−; 10, Cu2+; 11, Mg2+; 12, AlCl3; 13, Fe3+14, Fe2+; 15, CaCl2; 16, H2CO3; 17, Cys; 18, Cr3+; 19, Hsg;

FIG. 8 shows the effect of the probe TzAr-Nap-OH on the viability of HeLa cells; HeLa cells were treated with 0, 1, 2, 3, 5, 10 and 20 (μM) TzAr-Nap-OH for 24 hours, and the HeLa cell viability was determined by MTT; and

FIG. 9 is a fluorescence image of Hela cells; (a-c) HeLa cells were cultured with TzAr-Nap-OH (10 μM) for 30 minutes; (d-f) HeLa cells were treated with monensin (Mo) and incubated with the TzAr-Nap-OH probe for 30 minutes; λₑₓ=360 nm, slit=5 nm, scale bar=20 μm.

### DETAILED DESCRIPTION OF EMBODIMENTS

In order to further illustrate the present invention, the solutions provided by the present invention are described in detail below in conjunction with the drawings and examples, but they should not be understood as limiting the protection scope of the present invention.

### Embodiment 1

### Synthesis of Probe TzAr-Nap-OH

2,4-diphenyl-6-methyl-1,3,5-triazine (0.2 mmol), p-hydroxynaphtaldehyde (0.2 mmol), potassium hydroxide (1.0 mmol) and methanol (1.0 mL) were added in sequence into a 10 mL reaction tube and stirred at 100° C. for 24 hours. The solution was then cooled to room temperature and extracted three times with ethyl acetate. The organic phase was collected, dried over anhydrous sodium sulfate, and filtered with suction. The resulting filtrate was concentrated under reduced pressure and purified by column chromatography (dichloromethane:ethyl acetate=80:1), to obtain a yellow-green product TzAr-Nap-OH (15.15 mg, 48%) 1H NMR (600 MHZ, DMSO-d6): δ 10.07 (s, 1H), 8.71-8.69 (m, 4H), 8.57 (d, J=15.8 Hz, 1H), 8.25 (s, 1H), 7.99-7.96 (m, 1H), 7.88 (d, J=8.8 Hz, 1H), 7.78 (d, J=8.6 Hz, 1H), 7.73-7.69 (m, 2H), 7.68-7.64 (m, 4H), 7.40 (d, J=15.8 Hz, 1H), 7.21-7.15 (m, 2H); 13C NMR (150 MHz, DMSO-d6): δ 172.3, 171.0, 167.4, 157.3, 143.2, 136.2, 135.9, 133.3, 132.2, 132.0, 130.9, 130.8, 130.0, 129.4, 129.0, 128.0, 127.4, 124.9, 124.8. 119.8, 109.5;

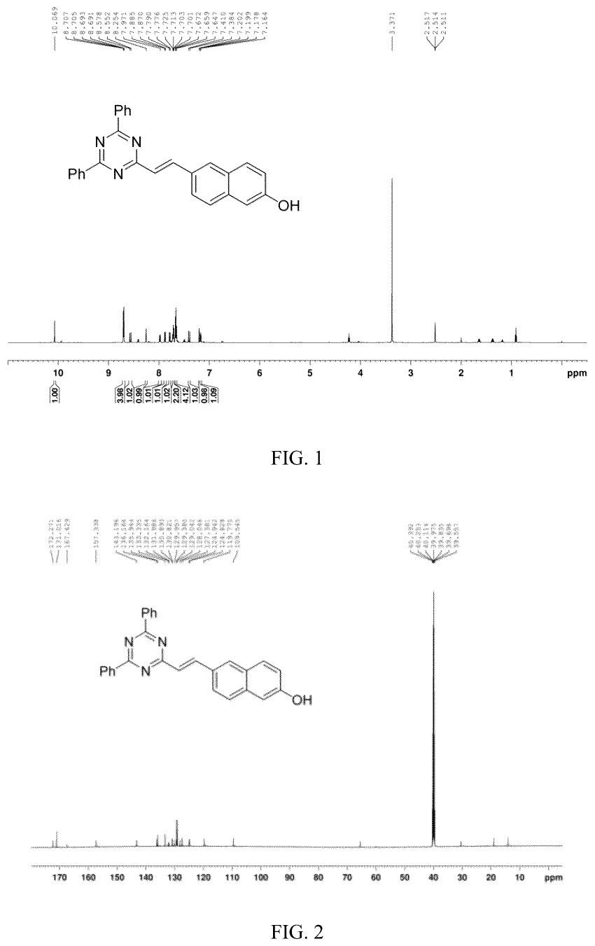

The 1H NMR spectrum of the probe is shown in FIG. 1, and the 13C NMR spectrum of the probe is shown in FIG. 2.

The molecular formula of the probe TzAr-Nap-OH is: C27H19N3O.

### Embodiment 2

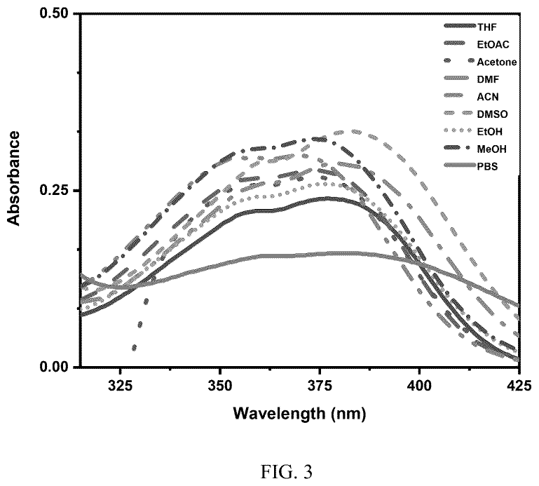

The fluorescent probe TzAr-Nap-OH was dissolved in dimethyl sulfoxide (DMSO) to prepare a 1 mmol/L stock solution. 20 μL probes were added dropwise in different solvents, with a final volume of 2 mL, and UV absorption spectra of the probes were measured, as shown in FIG. 3. The maximum absorption peak of TzAr-Nap-OH is approximately 360 nm.

### Embodiment 3

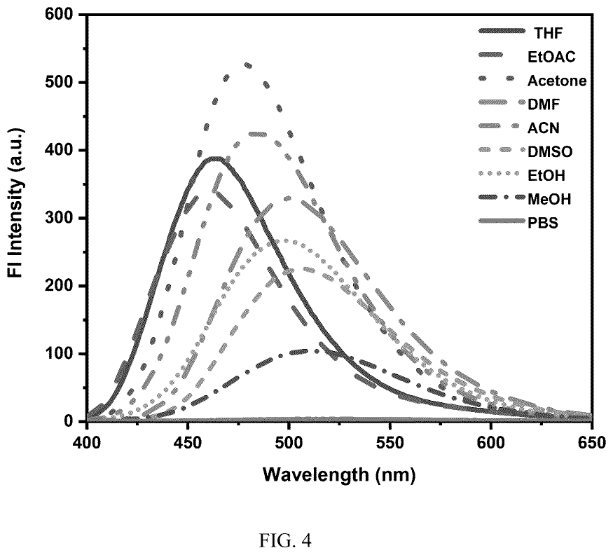

20 μL of the fluorescent probe stock solution prepared in Example 2 were taken, and added dropwise to different solvents, resulting in a final volume of 2 mL. The fluorescence emission spectra of the probe is measured (λₑₓ=360 nm), as shown in FIG. 4.

### Embodiment 4

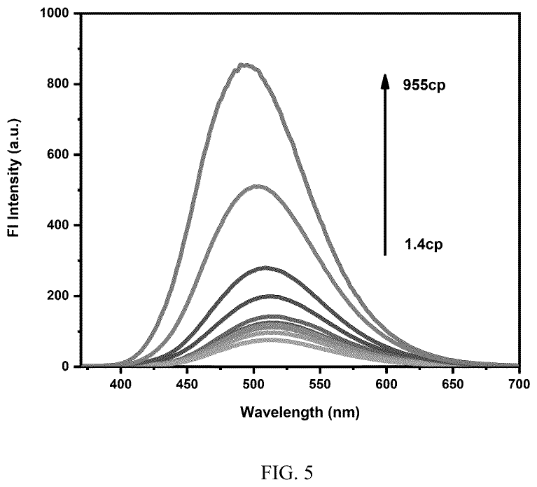

The spectral response of the probe to solution viscosity was measured, as shown in FIG. 5. By adjusting a volume ratio of glycerol to MeOH to change the viscosity of the system, the viscosity of the solution was increased from 1.4 cp to 955 cp, and it may be found that the fluorescence intensity at 520 nm was increased by nearly 15 times, which was because the rotation of molecules was hindered by the viscous environment, the non-radiative path was restricted and the fluorescence intensity was increases accordingly. Therefore, it may be concluded that the fluorescence intensity of TzAr-Nap-OH may be used as a reliable indicator for a quantitative viscosity sensor.

### Embodiment 5

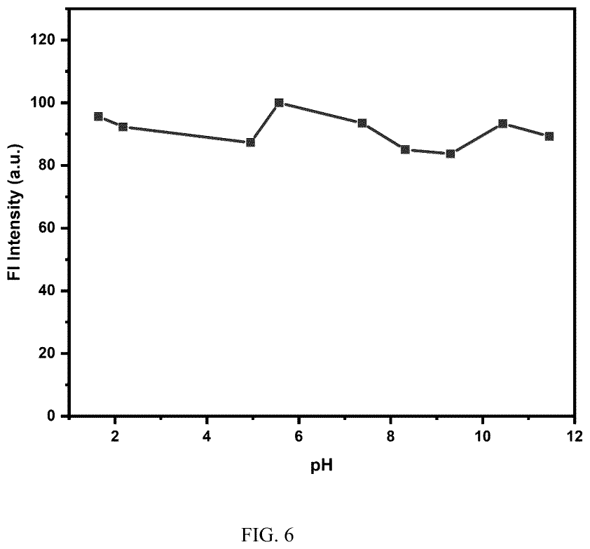

20 μL of the fluorescent probe stock solution were taken and added dropwise into a PBS/THF (4/1) system, resulting in a final volume of 2 mL. The effects of different pH of the solution on the probe were measured, as shown in FIG. 6. The fluorescence intensity of the probe did not change significantly within a wide pH range of 1.64 to 11.45, indicating that the probe TzAr-Nap-OH has good stability in acid-base physiological environments and may be applied in complex biological environments.

### Embodiment 6

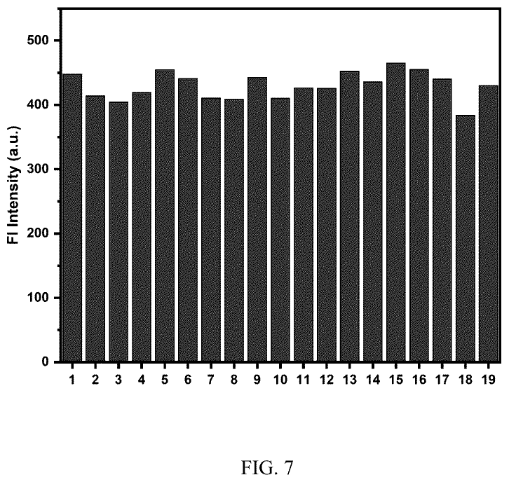

20 μL of the fluorescent probe stock solution were taken and added dropwise into the PBS/THF (4/1) system, into which 10 μM of other interfering substance solutions were added dropwise, resulint in a final volume is 2 mL. The effects of the interfering substances on TzAr-Nap were measured, as shown in FIG. 7. Other interfering substances had little effect on the fluorescence intensity of TzAr-Nap-OH.

### Embodiment 7

Cytotoxicity experiments were performed on TzAr-Nap-OH, as shown in FIG. 8. MTT analysis of TzAr-Nap-OH in Hela cells showed a survival rate of over 81%, indicating that the probe may serve as a practical tool for viscosity labeling in complex biological environments.

### Embodiment 8

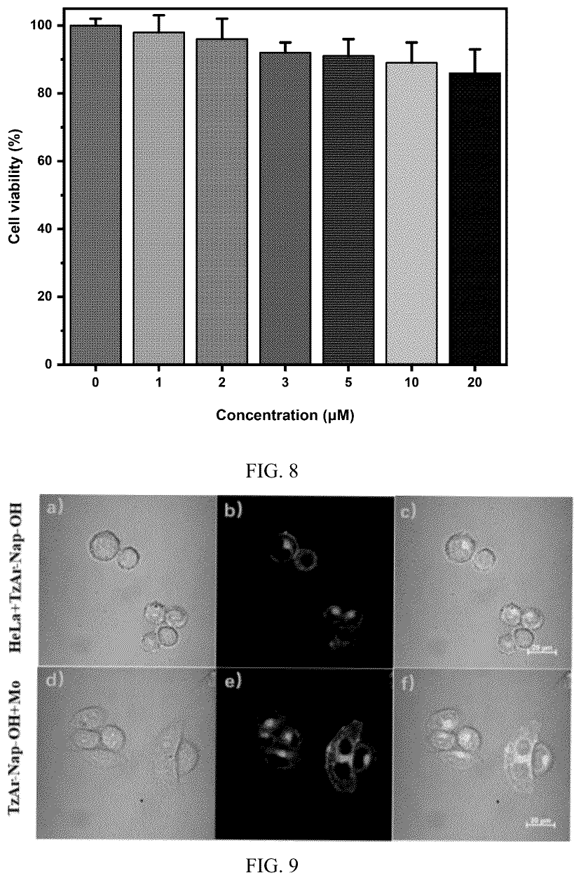

Cell imaging of TzAr-Nap-OH response to viscosity was shown in FIG. 9. Images a-c were obtained by treating HeLa cells with TzAr-Nap-OH (10 μM) for 30 mins. HeLa cells were first treated with monensin to increase cell viscosity, and then treated with TzAr-Nap-OH (10 μM) to obtain images d-f. From the comparison of bright field images a and d, the cells treated with monensin showed significant morphological changes, with significant swelling and increased viscosity; from the comparison of dark field images b and e, the fluorescence intensity of the cells treated with monensin was significantly higher than that of normal cells; and images c and f were mixed images of bright and dark fields, showing cell position and fluorescence intensity. These data demonstrated that the probe TzAr-Nap-OH may be precisely localized in cells and may be a good cell marker for cell viscosity response.

Although the present invention has been described in detail with reference to the above embodiments, it is only a part but not all the embodiments of the present invention, and other embodiments may be obtained without inventive step according to the embodiments, which all fall within the scope of the present invention.

What is claimed is:

1. A viscosity fluorescent probe, wherein the viscosity fluorescent probe has a molecular formula of C27H19N3O.

2. The viscosity fluorescent probe according to claim 1, wherein the molecular structural formula is as shown in Formula I:

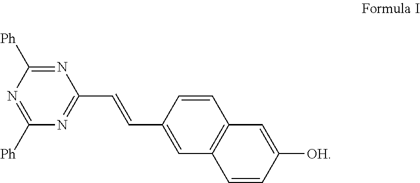 [SMILES: Oc1ccc2cc(/C=C/c3nc(-c4ccccc4)nc(-c4ccccc4)n3)ccc2c1]

3. The preparation method of the viscosity fluorescent probe according to claim 2, comprising the following steps: mixing 2,4-diphenyl-6-methyl-1,3,5-triazine, p-hydroxynaphthaldehyde, potassium hydroxide and methanol, and stirring at 100° C. for 24 hours;
cooling the mixed liquid, extracting with ethyl acetate, collecting an organic phase, drying with anhydrous sodium sulfate, and filtering with suction, concentrating the obtained filtrate under reduced pressure, and purifying same to obtain the viscosity fluorescent probe.

4. The preparation method according to claim 3, wherein during the mixing, a molar ratio of 2,4-diphenyl-6-methyl-1,3,5-triazine, p-hydroxynaphthaldehyde and potassium hydroxide is 0.2:0.2:1.

5. The preparation method according to claim 3, wherein the mixture is extracted three times with ethyl acetate.

6. The preparation method according to claim 3, wherein the purification comprises column chromatography purification.

7. Application of the viscosity fluorescent probe according to claim 1 in detection of viscosity in biological cell systems.

8. The application according to claim 7, wherein the detection comprises sensing detection.

9. The application according to claim 8, wherein the detection comprises fluorescence detection and cell imaging detection.
